(19) United States
(12) Patent Application Publication
(10) Pub. No.: US 2010/0104502 A1
(43) Pub. Date: Apr. 29, 2010

(54) COMPOSITIONS AND METHODS FOR DETECTING AND TREATING ENDOTHELIAL DYSFUNCTION

(75) Inventors: Daniel T. Eitzman, Saline, MI (US); Peter F. Bodary, New Boston, MI (US); Jonaton W. Homeister, Releieh, NC (US)

Correspondence Address: Casimir Jones, S.C., 2275 DEMING WAY, SUITE 310, MIDDLETON, WI, 53562, US

(73) Assignee: THE REGENTS OF THE UNIVERSITY OF MICHIGAN, Ann Arbor, MI (US)

(21) Appl. No.: 12/513,826

(22) PCT Filed: Nov. 6, 2007
(86) PCT No.: PCT/US07/83792

§ 371 Date: Dec. 2, 2009

Related U.S. Application Data
(60) Provisional application No. 60/856,995, filed on Nov. 6, 2006.

(51) Int. Cl. G01N 33/53; A61K 51/00; A61K 49/00; A61K 49/06; A61K 49/04; C12Q 1/68

(52) U.S. Cl. 424/1.11; 424/9.1; 424/9.3; 424/9.4; 435/6; 435/7.1; 435/7.92; 435/7.94

(57) ABSTRACT

The present invention relates to endothelial dysfunction. In particular, the present invention provides biomarkers of endothelial dysfunction (e.g., vascular disease), and compositions and methods of using the same. Compositions and methods of the present invention find use in, among other things, research, clinical, diagnostic, drug discovery, and therapeutic applications.

This application is a national stage application under 35 U.S.C. §371 of International Application PCT/US2007/083792, filed Nov. 6, 2007, which claims priority to U.S. Provisional Patent Application Ser. No. 60/856,995 filed Nov. 6, 2006, hereby incorporated by reference in its entirety.

This invention was made with government support under HL057346 awarded by National Heart, Lung, and Blood Institute. The government has certain rights in the invention.

### FIELD OF THE INVENTION

The present invention relates to endothelial dysfunction. In particular, the present invention provides biomarkers of endothelial dysfunction (e.g., vascular disease), and compositions and methods of using the same. Compositions and methods of the present invention find use in, among other things, research, clinical, diagnostic, drug discovery, and therapeutic applications.

### BACKGROUND OF THE INVENTION

Diseases of the vascular system remain the leading cause of mortality and morbidity in developed countries despite considerable therapeutic progress in recent years (See, e.g., Boersma et al., Lancet. 2003; 361: 847-858). Atherosclerosis is the predominant pathology underlying clinical vascular disease and comprises an intima-media plaque in conduit arteries containing cholesterol and inflammatory cells (See, e.g., Mullenix et al., Ann Vasc Surg. 2005; 19: 130-138; Libby, Nature. 2002; 420: 868-874). The implications of inflammation for the development and progression of atherosclerosis have become increasingly evident, and thus the mechanisms leading to inflammatory leukocyte recruitment are of central importance (See, e.g., Libby, Nature. 2002; 420: 868-874; Libby, Am J Cardiol. 2000; 86: 3J-9J).

The selectins (P, E and L) are a class of adhesion molecules that play important roles in many physiological processes, including leukocyte rolling and adhesion on endothelial cells (See, e.g., Bevilacqua and Nelson. 1993. J. Clin. Invest 91:379-387). For example, P-selectin is a member of the selectin family and is localized in the membranes of the α-granules of platelets and the Weibel-Palade bodies of endothelial cells (See, e.g., Blann et al., Eur Heart J. 2003; 24: 2166-2179; McEver R P. Selectins Curr Opin Immunol. 1994; 6: 75-84). P-selectin is expressed on the surface of activated platelets and endothelial cells and is important for leukocyte recruitment to sites of vascular injury and inflammation by engaging its ligand P-selectin glycoprotein ligand-1 (PSGL-1, See, e.g., Furie et al., Trends Mol Med. 2004; 10: 171-178).

P-selectin glycoprotein ligand-1 (PSGL-1), expressed primarily on leukocytes (See, e.g., McEver and Cummings. 1997. J. Clin. Invest 100:485-491) requires α(1,3)-fucosylation for binding activity (See, e.g., Homeister et al., 2001 Immunity. 15:115-126; Huang et al., 2000. J. Biol. Chem. 275:31353-31360). Adhesive interactions between selectins and PSGL-1 facilitate leukocyte rolling on endothelial cells (See, e.g., Norman et al., 1995. Blood 86:4417-4421; Moore et al., 1995. J. Cell Biol. 128:661-671) mediate the formation of platelet-leukocyte aggregates (See, e.g., Huo et al., 2003. Nat. Med. 9:61-67) and also transduces intracellular signals (See, e.g., McEver, Ann NY Acad Sci. 1994; 714: 185-189; McEver et al., Agents Actions Suppl. 1995; 47: 117-119).

Elevated levels of soluble selectins are associated with disease processes that involve selectin/selectin-ligand interactions. Thus, a need exists to better understand the regulation of selectin expression and the generation of soluble selectin, and for agents capable of altering such expression and/or generation.

### SUMMARY OF THE INVENTION

The present invention relates to endothelial dysfunction. In particular, the present invention provides biomarkers of endothelial dysfunction (e.g., vascular disease), and compositions and methods of using the same. Compositions and methods of the present invention find use in, among other things, research, clinical, diagnostic, drug discovery, and therapeutic applications.

Accordingly, in some embodiments, the present invention provides a method for detecting endothelial dysfunction in a subject, comprising providing a subject; and detecting expression and/or activity of P-selectin glycoprotein ligand 1 (PSGL-1) in the subject. The present invention is not limited by the methods of detecting PSGL-1 expression and/or activity. Indeed a variety of methods can be used including, but not limited to, RT-PCR, gene chip, radioimmunoassay, ELISA or other qualitative assay described herein. In some embodiments, detecting PSGL-1 comprises detecting PSGL-1 nucleic acid. In some embodiments, detecting PSGL-1 comprises detecting PSGL-1 protein. In some embodiments, detecting PSGL-1 protein comprises detecting fucosylation of PSGL-1. In some embodiments, detecting endothelial dysfunction in a subject further comprises detecting soluble P-selectin. In some embodiments, detecting endothelial dysfunction in a subject further comprises detecting soluble E-selectin. In some embodiments, detecting endothelial dysfunction in a subject further comprises detecting expression of one or more additional proteins. The present invention is not limited to the other types of proteins detected. Indeed, a variety of other proteins may be detected including, but not limited to, soluble ICAM-1, soluble VCAM-1, soluble thrombomodulin, and von Willebrand factor. In some embodiments, the expression and/or activity of PSGL-1 in the subject is elevated compared to a healthy subject. In some embodiments, endothelial dysfunction is associated with a disease including, but not limited to, hypertension, hypercholesterolaemia, vascular disease and diabetes. Different types of vascular disease can be detected included, but not limited to, atherosclerosis, artery disease, vascular disease, cardiovascular disease, restinosis, stenosis, occlusion, hemostatic disorder, coronary artery disease, stroke, heart attack, and/or diabetes mellitus. In some embodiments, detecting characterizes leukocyte interactions with endothelial cells. In some embodiments, detecting is used to monitor ligand-selectin interactions in vivo.

The present invention also provides a method for characterizing the efficacy of therapeutic drug treatment comprising: providing a subject; determining the expression level of PSGL-1 in the subject prior to the treatment; administering the treatment to the subject; and determining the expression level of PSGL-1 in the subject subsequent to the treatment. In some embodiments, the subject is a subject at risk for endothelial dysfunction or a subject suffering from endothelial dysfunction. In some embodiments, the subject is a healthy subject (e.g., displays no signs or symptoms of disease). In some embodiments, the method further comprises detecting the level of soluble P selectin in the subject prior to the treatment. In some embodiments, the method further comprises detecting the level of soluble P selectin in the subject subsequent to the treatment. In some embodiments, the method further comprises detecting the level of soluble E selectin in the subject prior to the treatment. In some embodiments, the method further comprises detecting the level of soluble E selectin in the subject subsequent to the treatment. In some embodiments, detection of a decrease in levels of soluble P selectin and/or soluble E selectin in the subject subsequent to the treatment is indicative of a favorable response to the treatment.

The present invention also provides a method of characterizing leukocyte interactions with endothelial cells or platelets in a subject comprising detecting levels of PSGL-1 in the subject. In some embodiments, the method further comprises detecting the expression of a soluble selectin (e.g., soluble P-selectin, soluble E-selectin and/or soluble L-selectin). In some embodiments, leukocyte interactions with endothelial cells or platelets are associated with endothelial dysfunction.

The present invention also provides a method for determining a course of treatment in a subject comprising: providing a subject; determining the expression level of PSGL-1 in the subject; and identifying a course of treatment for the subject based upon the expression level of PSGL-1 in the subject. In some embodiments, the method further comprises determining the expression level of PSGL-1 in the subject subsequent to administering the treatment and/or during the course of treatment to the subject. In some embodiments, the subject is a subject at risk for endothelial dysfunction and/or a subject suffering from endothelial dysfunction. In some embodiments, the method further comprises detecting the level of soluble P selectin and/or soluble E selectin in the subject. In some embodiments, the method further comprises determining the expression level of soluble P selectin and/or soluble E selectin in the subject subsequent to administering the treatment and/or during the course of treatment to the subject. In some embodiments, detection of a decrease in levels of soluble P selectin and/or soluble E selectin in the subject subsequent to the treatment is indicative of a favorable response to the treatment. In some embodiments, the course of treatment is a treatment for a disease including, but not limited to, hypertension, hypercholesterolaemia, vascular disease and/or diabetes. In some embodiments, the vascular disease is atherosclerosis, artery disease, vascular disease, cardiovascular disease, restinosis, stenosis, occlusion, hemostatic disorder, coronary artery disease, stroke, heart attack, and/or diabetes mellitus.

### DESCRIPTION OF THE DRAWINGS

FIG. 1 shows the effect of PSGL-1 or FucT-VII deficiency on sP-sel levels. A) sP-sel levels in Psgl-1^{+/+} mice compared to Psgl-1^{-/-} mice. *p<0.00001. B) sP-sel levels in FucT-VII^{+/+} mice compared to FucT-VII^{-/-} mice (n=6). *p<0.00001.

FIG. 2 shows the effect of PSGL-1 or FucT-VII deficiency on sE-sel levels. A) sE-sel levels in Psgl-1^{+/+} mice compared to Psgl-1^{-/-} mice. *p<0.00001. B) sE-sel levels in FucT-VII^{+/+} mice compared to FucT-VII^{-/-} mice. *p<0.001.

FIG. 3 shows the effect of bone marrow transplantation on sP and E-sel levels. A) sP-sel levels in Psgl-1^{+/+} mice transplanted with Psgl-1^{+/+} or Psgl-1^{-/-} bone marrow. *p<0.00001 B) sE-sel levels in Psgl-1^{+/+} mice transplanted with Psgl-1^{+/+} or Psgl-1^{-/-} bone marrow. *p<0.001.

### DEFINITIONS

As used herein, the term “animal” refers to any animal (e.g., a mammal), including, but not limited to, humans, non-human primates, rodents (e.g., mice, rats, etc.), flies, and the like.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)₂ fragments, and Fab expression libraries; and single chain antibodies.

The term “epitope” as used herein refers to that portion of an antigen that makes contact with a particular immunoglobulin.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “specifically binding to PSGL-1 with low background binding” refers to an antibody that binds specifically to PSGL-1 protein (e.g., in an immunohistochemistry assay) but not to other proteins (e.g., lack of non-specific binding).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “endothelial dysfunction” refers generally to a physiological dysfunction of normal biochemical processes carried out by the endothelium, the cells that line the inner surface of all blood vessels including arteries and veins as well as the innermost lining of the heart and lymphatics. Thus, a compromise of normal function of endothelial cells is characteristic of endothelial dysfunction (e.g., characterized by the inability of arteries and arterioles to dilate fully in response to an appropriate stimulis). Normal functions of endothelial cells include, but are not limited to, mediation of coagulation, platelet adhesion, immune function, control of volume and electrolyte content of the intravascular and extravascular spaces. Endothelial dysfunction can result from a multitude of factors including, but not limited to, disease processes (e.g., hypertension, hypercholesterolaemia, diabetes) as well as from environmental factors (e.g., such as from smoking tobacco products). Thus, the term “endothelial dysfunction” also relates generally to the development of vascular disease (e.g., including, but not limited to, atherosclerosis, artery disease, vascular disease, cardiovascular disease, restinosis, stenosis, occlusion, abnormal leukocyte recruitment, abnormal cell to cell adhesion, abnormal cell adhesion to blood vessels, inflammation, hemostatic disorders (e.g., hemorrhagic and/or thrombotic disorders), coronary artery disease, stroke, heart attack, and diabetes mellitus) as signs and symptoms of endothelial dysfunction can be observed to predate clinically detectable vascular pathology (e.g., associated with one or more vascular diseases (e.g., those described above) by many years).

As used herein, the term “subject is suspected of having endothelial dysfunction” refers to a subject that presents one or more symptoms indicative of a medically relevant endothelial dysfunction (e.g., caused by a disorder, disease (e.g., vascular disease (e.g., atherosclerosis)), aging, genetic predisposition, or injury). A subject suspected of having endothelial dysfunction has generally not been tested for endothelial dysfunction. However, a “subject suspected of having endothelial dysfunction” encompasses an individual who has received a preliminary diagnosis but for whom a confirmatory test has not been done or for whom the degree of endothelial dysfunction is not known. A “subject suspected of having endothelial dysfunction” is sometimes diagnosed with endothelial dysfunction and is sometimes found to not have endothelial dysfunction.

As used herein, the term “subject diagnosed with a endothelial dysfunction” refers to a subject who has been tested and found to have endothelial dysfunction. Examples of such subjects include, but are not limited to, subjects with vascular disease (e.g., atherosclerosis).

As used herein, the term “subject at risk for endothelial dysfunction” refers to a subject with one or more risk factors for developing endothelial dysfunction. Risk factors include, but are not limited to, gender, age, genetic predisposition (e.g., genetic disorder), environmental exposure, and previous incidents of diseases, and lifestyle.

As used herein, the term “characterizing endothelial dysfunction” refers to the identification of one or more characteristics of a subject suspected as having endothelial dysfunction, diagnosed as having endothelial dysfunction or at risk for endothelial dysfunction including, but not limited to, the expression and/or activity of PSGL-1, the expression and/or activity of soluble P selectin and/or soluble E selectin. Thus, endothelial dysfunction may be characterized by the characterization of the expression level of one or more biomarkers (e.g., PSGL-1, soluble P selectin and/or soluble E selectin) in the subject.

As used herein, the term “characterizing tissue in a subject” refers to the identification of one or more properties of a tissue sample (e.g., including but not limited to, morphology and cellular localization). In some embodiments, tissues are characterized by the identification of the expression level of one or more biomarkers (e.g., PSGL-1, soluble P selectin and/or soluble E selectin) in the tissue.

As used herein, the term “reagent(s) capable of specifically detecting biomarker expression” refers to reagents used to detect (e.g., sufficient to detect) the expression of biomarkers of the present invention (e.g., PSGL-1, soluble P selectin and/or soluble E selectin). Examples of suitable reagents include, but are not limited to, nucleic acid probes capable of specifically hybridizing to biomarker mRNA or cDNA, and antibodies.

As used herein, the term “instructions for using said kit for detecting endothelial dysfunction” includes instructions for using the reagents contained in the kit for the detection and characterization of endothelial dysfunction in a sample (e.g., derived from a subject).

As used herein, the term “effective amount” refers to the amount of a composition (e.g., inducer of PSGL-1, soluble P selectin and/or soluble E selectin expression and/or activity) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent (e.g., a test compound), or therapeutic treatment to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

As used herein, the term “co-administration” refers to the administration of at least two agent(s) (e.g., a test compound and one or more other agents—e.g., PSGL-1) or therapies to a subject (e.g., a human or mouse). In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., test compound) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “gene transfer system” refers to any means of delivering a composition comprising a nucleic acid sequence (e.g., encoding PSGL-1) to a cell or tissue.

For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1-3 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (e.g., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (e.g., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (e.g., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent (50×Denhardt's contains per 500 ml: 5 g Ficoll (Type 400, Pharamcia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., endothelial dysfunction). Test compounds comprise both known and potential therapeutic compounds. A test compound can be determined to be therapeutic by screening using the screening methods of the present invention. Examples of test compounds include, but are not limited to, carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, drug, antibody, prodrug, glycopeptides, glycoproteins, proteoglycans and the like, and synthetic analogues or derivatives thereof, including peptidomimetics, small molecule organic compounds and the like, and mixtures thereof (e.g., that is a candidate for use to treat or prevent a disease, illness, sickness, or disorder of bodily function (e.g., endothelial dysfunction). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “entering” as in “entering said PSGL-1 activity and/or expression level information into said computer” refers to transferring information to a “computer readable medium.” Information may be transferred by any suitable method, including but not limited to, manually (e.g., by typing into a computer) or automated (e.g., transferred from another “computer readable medium” via a “processor”).

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “computer implemented method” refers to a method utilizing a “CPU” and “computer readable medium.”

### DETAILED DESCRIPTION OF THE INVENTION

The present invention relates to endothelial dysfunction. In particular, the present invention provides biomarkers of endothelial dysfunction (e.g., vascular disease), and compositions and methods of using the same. Compositions and methods of the present invention find use in, among other things, research, clinical, diagnostic, drug discovery, and therapeutic applications.

Endothelial dysfunction is a key event in cardiovascular disease. Measurement of endothelial dysfunction in vivo presents a major challenge, but has important implications since it may identify the clinical need for therapeutic intervention.

The ability of cells to adhere to one another plays a critical role in development, normal physiology, and disease processes. This ability is mediated by adhesion molecules, generally glycoproteins, expressed on the cell surface. Several important classes of adhesion molecules include the integrins, the selectins, and members of the immunoglobulin (Ig) superfamily. Selectins play a central role in mediating leukocyte adhesion to activated endothelium and platelets.

Blood clotting, along with inflammation and tissue repair, are host defense mechanisms which function in parallel to preserve the integrity of the vascular system after tissue injury. In response to tissue injury, platelets, endothelial cells and leukocytes are important for the formation of a platelet plug, deposition of leukocytes in injured tissue, initiation of inflammation, and wound healing.

The selectins (P, E and L) are a class of adhesion molecules that play important roles in many physiological processes, including leukocyte rolling and adhesion on endothelial cells (See, e.g., Bevilacqua and Nelson. 1993. J. Clin. Invest 91:379-387). P-selectin (P-sel) is stored in platelet α-granules and endothelial cell Weibel-Palade bodies and is rapidly expressed on the cell surface following stimulation (See, e.g., Stenberg et al., 1985. J. Cell Biol. 101:880-886; Stenberg et al., 1985. J. Cell Biol. 101:880-886). In addition to its role in leukocyte rolling and extravasation in inflammation, P-selectin mediates platelet-leukocyte adhesion within thrombi, and increases tissue factor expression on monocytes, thereby promoting fibrin deposition by leukocytes and thrombogenesis (Palabrica, T. et al. Nature (1992) 359:848-851; Celi, A. et al. Proc Natl Acad Sci USA (1994) 91:8767-8771). E-selectin (E-sel) is expressed on endothelial cells following cytokine stimulation (See, e.g., Bevilacqua et al., 1989. Science 243:1160-1165) and L-selectin (L-sel) is expressed on leukocytes (See, e.g., Schleiffenbaum et al., 1992. J. Cell Biol. 119:229-238). Deficiency of one or more of these selectins has been shown to alter vascular disease processes in several preclinical models (See, e.g., Etzioni et al., 1999. Blood 94:3281-3288).

Although the membrane-bound selectins mediate cell-cell interactions in the vasculature, each selectin also has a soluble form (sE-sel, sP-sel, sL-sel) that can be measured in the plasma (See, e.g., Schleiffenbaum et al., 1992. J. Cell Biol. 119:229-238; Andre et al., 2000. Proc. Natl. Acad. Sci. U.S. A 97:13835-13840; Ruchaud-Sparagano et al., 2000. J. Biol. Chem. 275:15758-15764). The circulating soluble selectins have been used as markers of vascular disease processes (See, e.g., Atalar et al., 2001. Int. J. Cardiol. 78:69-73) and may play direct roles in inflammatory disease processes (See, e.g., Schleiffenbaum et al., 1992. J. Cell Biol. 119:229-238; Andre et al., 2000. Proc. Natl. Acad. Sci. U.S. A 97:13835-13840; Ruchaud-Sparagano et al., 2000. J. Biol. Chem. 275:15758-15764).

The cell surface expression of P-selectin is tightly regulated, and P-selectin is rapidly shed from the cell surface upon platelet activation, appearing as a soluble fragment in the plasma (Berger, G. et al. Blood (1998) 92:4446-4452). Soluble P-selectin may also result from an alternatively spliced isoform of P-selectin lacking the transmembrane domain (Ishiwata, N. et al. J. Biol Chem (1994) 269:23708). The plasma of healthy humans and mice contains little soluble P-selectin, as detected by ELISA, and an increase in plasma P-selectin concentration may indicate in vivo activation of and/or damage to platelets and endothelial cells.

A physiologically important endogenous ligand for the selectins is P-selectin glycoprotein ligand-1 (PSGL-1), that is expressed primarily on leukocytes (See, e.g., McEver and Cummings. 1997. J. Clin. Invest 100:485-491) and requires α(1,3)-fucosylation for binding activity (See, e.g., Homeister et al., 2001. Immunity. 15:115-126; Huang et al., 2000. J. Biol. Chem. 275:31353-31360). Adhesive interactions between selectins and PSGL-1 facilitate leukocyte rolling on endothelial cells (See, e.g., Norman et al., 1995. Blood 86:4417-4421; Moore et al., 1995. J. Cell Biol. 128:661-671) and mediate the formation of platelet-leukocyte aggregates (See, e.g., Huo et al., 2003. Nat. Med. 9:61-67). Deficiency of PSGL-1 in mice has been associated with impaired leukocyte rolling and reduced generation of procoagulant microparticles (See, e.g., Yang et al., 1999. J. Exp. Med. 190:1769-1782; Hrachovinova et al., 2003. Nat. Med. 9:1020-1025).

Because elevated levels (e.g., compared to a healthy, normal or control subject (e.g., over a period of time (e.g., a day, a week, a month, a year or longer)) of soluble selectins are associated with disease processes which require selectin/selectin-ligand interactions, experiments were conducted during development of the present invention in order to characterize the generation of circulating soluble selectins. The present invention provides that leukocyte interactions with endothelial cells is important in the generation of sE-sel and sP-sel and that PSGL-1 and alpha(1,3)-fucosyltransferase activity is important for sE-sel and sP-sel levels (See, e.g., Examples 1-5). The present invention also provides that the level of soluble selectins can be used as specific biomarkers of leukocyte interactions with endothelial cells and can be used to track ligand-selectin interactions (e.g., in vitro, in vivo or ex vivo) (See, e.g., Examples 1-5). For example, the presence (e.g., expression and/or activity) of one or more biomarkers of the present invention (e.g., sP-sel, sE-sel, sL-sel and/or PSGL-1 (e.g., in the circulation) can be used to identify an ongoing physiological process (e.g., endothelial dysfunction). For example, the levels (e.g., of expression and/or activity) of one or more biomarkers (e.g., compared to the levels of the one or more biomarkers in a healthy, normal and/or control subject) present in a subject can be used as a marker to identify endothelial dysfunction in a subject. For example, a subject that displays elevated levels (e.g., compared to a healthy, normal and/or a control subject (e.g., over a period of time (e.g., a day, a week, a month, a year or longer)) of one or more biomarkers of the present invention may be identified as a subject with endothelial dysfunction (e.g., vascular disease).

### I. Biomarkers for Endothelial Dysfunction

The present invention provides biomarkers (e.g., sP-sel, sE-sel, sL-sel and/or PSGL-1) whose presence and/or expression is specifically detectable and/or altered during endothelial dysfunction (e.g., associated with vascular disease (e.g., including, but not limited to, atherosclerosis, artery disease, vascular disease, cardiovascular disease, restinosis, stenosis, occlusion, abnormal leukocyte recruitment, abnormal cell to cell adhesion, abnormal cell adhesion to blood vessels, inflammation, hemostatic disorders (e.g., hemorrhagic and/or thrombotic disorders), coronary artery disease, stroke, heart attack, and diabetes mellitus)). Such biomarkers find use in the identification and characterization of endothelial dysfunction (e.g., for use in clinical and/or basic research applications).

A. Identification of Markers

The present invention provides a comprehensive view of genetic determinants (e.g., biomarkers) that specify endothelial dysfunction (e.g., PSGL-1, sP-sel, sE-sel and/or sL-sel). In particular, the present invention provides that the generation of sP-sel and sE-sel levels in vivo are dependent upon a requirement for bone marrow-derived PSGL-1 and α(1,3)-fucosyltransferase activity (See Examples 2-3). Thus, the present invention provides that PSGL-1 in addition to soluble selectins can be used as specific markers of leukocyte interactions with endothelial cells and platelets and serve as a valuable tool in tracking, monitoring and/or characterizing ligand-selectin interactions in vivo. The present invention also provides that the presence of persistent PSGL-1 and/or soluble adhesion molecules in the circulation is indicative of ongoing physiological processes (e.g., associated with endothelial dysfunction (e.g., vascular disease)) reflecting cell-cell interactions, and that selectin shedding plays an important regulatory role in leukocyte adhesive interactions with endothelial cells.

B. Biomarker Detection and Treatment Options

In some embodiments, the present invention provides methods for detection of expression of an endothelial dysfunction biomarker (e.g., PSGL-1, sP-sel, sE-sel and/or sL-sel). In some embodiments, expression is measured directly (e.g., at the nucleic acid or protein level). In some embodiments, expression is detected in tissue samples (e.g., biopsy tissue). In other embodiments, expression is detected in bodily fluids. The present invention further provides panels and kits for the detection of biomarkers. In preferred embodiments, the presence of a biomarker is used to provide information related to endothelial dysfunction (e.g., vascular disease) presence and/or status to a subject. For example, the detection of PSGL-1, sP-sel, and/or sE-sel may be indicative of vascular disease that may benefit from a certain treatment compared to a disease lacking detectable biomarker expression and/or activity. In addition, the expression level of one or more biomarkers identified herein (e.g., PSGL-1, sP-sel, sE-sel and/or sL-sel) may be indicative of a type of vascular disease (e.g., atherosclerosis) in a subject.

The information provided can also be used to direct a course of treatment. For example, if a subject is found to possess or lacks a biomarker (e.g., PSGL-1, sP-sel, and/or sE-sel), therapies can be chosen to optimize the response to treatment.

The present invention is not limited to any particular biomarker. Indeed, any biomarker identified herein that correlates with endothelial dysfunction may be utilized, alone or in combination, including, but not limited to, PSGL-1, soluble P-sel, soluble E-sel and/or soluble L-sel (See Examples 2-5). Furthermore, post-translational modification status (e.g., fucosylation status) of a biomarker (e.g., fucosylation of PSGL-1) may also be detected. Additional biomarkers (e.g., sICAM-1 and sVCAM-1, soluble thrombomodulin, and/or von Willebrand factor) are also contemplated to be within the scope of the present invention for use with one or more of the biomarkers of the present invention. Any suitable method may be utilized to identify and characterize biomarkers suitable for use in the methods of the present invention including, but not limited to, those described in illustrative Examples 2-5 below. For example, in some embodiments, biomarkers identified as being up or down-regulated using the methods of the present invention are further characterized using microarray (e.g., nucleic acid or tissue microarray), immunohistochemistry, Northern blot analysis, siRNA or antisense RNA inhibition, mutation analysis, investigation of expression with clinical outcome, as well as other methods disclosed herein.

In some embodiments, the present invention provides a panel for the analysis of a plurality of biomarkers. The panel allows for the simultaneous analysis of multiple biomarkers correlating with endothelial dysfunction. For example, a panel may include biomarkers identified as correlating with the likelihood of a subject to respond to therapeutic treatment. Depending on the subject, panels may be analyzed alone or in combination in order to provide the best possible diagnosis and prognosis. Markers for inclusion on a panel are selected by screening for their predictive value using any suitable method including, but not limited to, those described in the illustrative examples below.

In other embodiments, the present invention provides an expression profile map comprising expression profiles of endothelial cells of various stages of disease development and/or activity. Such maps can be used for comparison with patient samples. Any suitable method may be utilized including, but not limited to, computer comparison of digitized data. The comparison data may be used for research purposes or to provide diagnoses and/or prognoses to patients. In some embodiments, detecting the expression and/or activity of one or more biomarkers of the present invention is utilized to characterize endothelial-leukocyte interaction, characterize general endothelial function, to assess (e.g., predict) risk of vascular events, and/or to characterize the efficacy of vascular disease therapies (e.g., existing as well as those in clinical trials and/or development (e.g., lipid lowering agents (e.g., statins))).

1. Detection of Nucleic Acids (e.g., DNA and RNA)

In some preferred embodiments, detection of biomarkers (e.g., including, but not limited to, those disclosed herein) is detected by measuring the levels of the biomarker (e.g., PSGL-1, sP-sel, sE-sel and/or sL-sel) in cells, tissue (e.g., endothelial cells and tissues) and/or serum. For example, in some embodiments, PSGL-1 can be monitored using antibodies (e.g., commercially available antibodies (e.g., from R&D Systems, Minneapolis, Minn., or generated according to methods described below) and/or by detecting PSGL-1 protein. In some embodiments, detection is performed on cells or tissue after the cells or tissues are removed from the subject. In other embodiments, detection is performed by visualizing the biomarker (e.g., PSGL-1) in cells and tissues residing within the subject. In some embodiments, serum levels of sP-sel, sE-sel and/or sL-sel are detected independently or together with the expression of PSGL-1 (e.g., using antibodies).

In some embodiments, detection of biomarkers (e.g., PSGL-1, sP-sel, sE-sel and/or sL-sel) is detected by measuring the expression of corresponding mRNA in a sample (e.g., a tissue or cell sample). mRNA expression may be measured by any suitable method, including but not limited to, those disclosed herein.

In some embodiments, RNA is detected by Northern blot analysis. Northern blot analysis involves the separation of RNA and hybridization of a complementary labeled probe.

In still further embodiments, RNA (or corresponding cDNA) is detected by hybridization to an oligonucleotide probe). A variety of hybridization assays using a variety of technologies for hybridization and detection are available. For example, in some embodiments, TAQMAN assay (PE Biosystems, Foster City, Calif.; See e.g., U.S. Pat. Nos. 5,962,233 and 5,538,848, each of which is herein incorporated by reference) is utilized. The assay is performed during a PCR reaction. The TAQMAN assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleotlytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In some embodiments, profiles from healthy endothelial cells can be compared with profiles from diseased edothelial cells. For example, in some embodiments, a profile from a single cell is generated (e.g., isolated from a cell biopsy). Such a profile may characterize the expression of all genes in the cell. In some embodiments, a profile characterizes the expression of a subset of the genes expressed in the cell (e.g., characterizes the expression of biomarkers identified herein). Thus, a gene chip or RT-PCR or other qualitative assay described herein or well known in the art could be used to generate a profile (e.g., for use in diagnostic or treatment settings).

2. Detection of Protein

In other embodiments, gene expression of biomarkers is detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein (e.g., against PSGL-1, sP-sel, sE-sel and/or sL-sel). The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to biomarkers is utilized.

In other embodiments, an immunoassay described in U.S. Pat. Nos. 5,599,677 and 5,672,480; each of which is herein incorporated by reference, is utilized.

3. Data Analysis

The present invention also provides methods of analyzing, processing and presenting data regarding detection using a biomarker of the present invention (e.g., correlating gene profile of a diseased photoreceptor to that of a healthy photoreceptor using the specific biomarkers described herein (e.g., to provide diagnostic information and/or treatment options).

In some embodiments, a computer-based analysis program is used to translate the raw data generated by the detection assay (e.g., the presence, absence, or amount of a given biomarker or biomarkers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information providers, medical personal, and subjects. For example, in some embodiments of the present invention, a sample (e.g., a biopsy, cell, serum, or other sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., degree of endothelial cell involvement (e.g., in a vascular disease) or the likelihood of responding to a particular treatment) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of biomarkers as useful indicators of a particular condition or stage of disease.

4. Kits

In yet other embodiments, the present invention provides kits for the detection and characterization of biomarkers. In some embodiments, the kits contain antibodies specific for a biomarker (e.g., PSGL-1, sP-sel, sE-sel and/or sL-sel), in addition to detection reagents and buffers. In other embodiments, the kits contain reagents specific for the detection of mRNA or cDNA (e.g., oligonucleotide probes or primers). In preferred embodiments, the kits contain all of the components necessary and/or sufficient to perform a detection assay, including all controls, directions for performing assays, and any necessary software for analysis and presentation of results.

5. In Vivo Imaging

In some embodiments, in vivo imaging techniques are used to visualize the expression of biomarkers in an animal (e.g., a human or non-human mammal). For example, in some embodiments, biomarker mRNA or protein is labeled using a labeled antibody specific for the biomarker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the biomarkers of the present invention are described herein.

The in vivo imaging methods of the present invention are useful in identifying cells that express the biomarkers of the present invention (e.g., diseased cells associated with vascular disease). In vivo imaging is used to visualize the presence of a biomarker indicative of disease status. Such techniques allow for identification and characterization without the use of a biopsy. The in vivo imaging methods of the present invention are also useful for providing prognoses to patients (e.g., likelihood to respond to therapeutic treatment).

In some embodiments, reagents (e.g., antibodies) specific for the biomarkers of the present invention are fluorescently labeled. The labeled antibodies can be introduced into a subject (e.g., parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 (1990) have described an optimized antibody-chelator for the radioimmunoscintographic imaging of tumors using Indium-111 as the label. Griffin et al., (J Clin One 9:631-640 (1991)) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 (1991)). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 (1980)) for In-111 and Tc-99m, and by Scheinberg et al. (Science 215:1511 (1982)). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 (1982)) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 (1978)) for plasma protein, and recently applied successfully by Wong et al. (J. Nucl. Med., 23:229 (1981)) for labeling antibodies. In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific biomarker of the present invention, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a biomarker of the present invention). When active, it leads to a reaction that emits light. A CCD camera and software is used to capture the image and analyze it.

### II. Antibodies

The present invention provides isolated antibodies. In preferred embodiments, the present invention provides monoclonal or polyclonal antibodies that specifically bind to either an isolated polypeptide comprised of at least five amino acid residues of the biomarkers described herein (e.g., PSGL-1, sP-sel, sE-sel and/or sL-sel). These antibodies find use in the diagnostic methods described herein.

An antibody against a biomarker of the present invention may be any monoclonal or polyclonal antibody, as long as it can recognize the biomarker. Antibodies can be produced by using a biomarker of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, biomarkers, as such, or together with a suitable carrier or diluent is administered to an animal (e.g., a mammal) under conditions that permit the production of antibodies. For enhancing the antibody production capability, complete or incomplete Freund's adjuvant may be administered. Normally, the biomarker is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 (1975)). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a biomarker of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a biomarker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to a hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a biomarker of the present invention (further including a gene having a nucleotide sequence partly altered) can be used as the immunogen. Further, fragments of the protein may be used. Fragments may be obtained by any method including, but not limited to expressing a fragment of the gene, enzymatic processing of the protein, chemical synthesis, and the like.

### III. Drug Screening

In some embodiments, the present invention provides drug screening assays (e.g., to screen for endothelial dysfunction altering compounds). The screening methods of the present invention utilize biomarkers identified using the methods of the present invention (e.g., including but not limited to PSGL-1, sP-sel, sE-sel and/or sL-sel).

For example, in some embodiments, the present invention provides a method of screening for a compound that alters (e.g., increases or decreases) the presence of biomarkers (e.g., PSGL-1, sP-sel, sE-sel and/or sL-sel). In some embodiments, candidate compounds are antisense agents (e.g., oligonucleotides) directed against biomarkers (e.g., PSGL-1, sP-sel, sE-sel and/or sL-sel) or proteins that interact with a biomarker (e.g., that inhibit biomarker activity). In other embodiments, candidate compounds are antibodies that specifically bind to a biomarker of the present invention (e.g., PSGL-1, sP-sel, sE-sel and/or sL-sel) or proteins that interact with a biomarker (e.g., that inhibit biomarker activity). The present invention is not limited by the type of candidate compound utilized. Indeed, a variety of candidate compounds may be tested including, but are not limited to, carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, drug, antibody, prodrug, glycopeptides, glycoproteins, proteoglycans and the like, and synthetic analogues or derivatives thereof, including peptidomimetics, small molecule organic compounds and the like, and mixtures thereof.

In some embodiments, test compounds are screened (e.g., characterized) for their ability to alter (e.g., enhance or inhibit) the ability of PSGL-1 to increase the presence of soluble selectins (e.g., sP-sel, sE-sel, etc.). In some embodiments, a test compound is administered (e.g., to a subject with endothelial dysfunction) prior to therapeutic treatment (e.g., administration of a statin) for a particular form of endothelial dysfunction (e.g., vascular disease). In some embodiments, a test compound is administered (e.g., to a subject to a subject with endothelial dysfunction) subsequent to therapeutic treatment. In some embodiments, a test compound is administered (e.g., to a subject to a subject with endothelial dysfunction) both prior to as well as after therapeutic treatment. In some embodiments, one or more types of test compounds are administered to a subject. In some embodiments, compositions and methods of the present invention are used to characterize the affect of other conditions (e.g., age, diet, environmental exposure, etc.) on endothelial cells (e.g., response to test compounds, secretion of soluble selectins, etc.).

In one screening method, test compounds are evaluated for their ability to alter biomarker presence, activity or expression by contacting a test compound with a cell (e.g., a cell expressing or capable of expressing biomarker nucleic acid and/or protein (e.g., an endothelial cell) and then assaying for the effect of the test compounds on the presence or expression of a biomarker. In some embodiments, the effect of candidate compounds on expression or presence of a biomarker is assayed for by detecting the level of biomarker mRNA expressed by the cell. mRNA expression can be detected by any suitable method.

In other embodiments, the effect of test/candidate compounds on expression or presence of biomarkers is assayed by measuring the level of polypeptide encoded by the biomarkers. The level of polypeptide expressed can be measured using any suitable method including, but not limited to, those disclosed herein.

Specifically, the present invention provides screening methods for identifying modulators, i.e., candidate or test compounds or agents (e.g., proteins, peptides, peptidomimetics, peptoids, small molecules or other drugs) that bind to or otherwise directly or indirectly affect biomarkers of the present invention, have an inhibitory (or stimulatory) effect on, for example, biomarker (e.g., PSGL-1, sP-sel, sE-sel and/or sL-sel) expression, biomarker activity or biomarker presence, or have a stimulatory or inhibitory effect on, for example, the expression or activity of a biomarker substrate. Compounds thus identified can be used to modulate the activity of target gene products (e.g., biomarker genes) either directly or indirectly in a therapeutic protocol, to elaborate the biological function of the target gene product, or to identify compounds that disrupt normal target gene interactions. Compounds that inhibit or enhance the activity, expression or presence of biomarkers are useful in the treatment of disorders, diseases or the like characterized by endothelial dysfunction.

In some embodiments, the present invention provides assays for screening test compounds that can change cell activity (e.g., the amount of soluble selectin generated and/or secreted from a cell). For example, PSGL-1 expression and/or activity can be used to determine if a cell contains or will generate or will secrete soluble selectins. In some embodiments, the present invention provides a method for screening a test compound for the ability of the test compound to alter physiology, signs and/or symptoms associated with endothelial dysfunction. For example, in some embodiments, a test compound is administered to a subject (e.g., a human subject or non-human subject (e.g., an animal (e.g., mouse))) with endothelial dysfunction and the ability of the test compound to alter endothelial dysfunction signs, symptoms and/or physiology is characterized. In some embodiments, the expression and/or activity of PSGL-1 is also characterized (e.g., before, during and/or after treatment). In some embodiments, the amount of soluble selectin (e.g., sP-sel, sE-sel and/or sL-sel) is also characterized (e.g., before, during and/or after treatment). Thus, the expression and/or activity of a biomarker of the present invention (e.g., PSGL-1, sP-sel, sE-sel and/or sL-sel) can be used to determine the efficacy of a test compound. For example, a test compound that is able to reduce the levels (e.g., compared to a subject that has not received the test compound) of soluble selectin (e.g., sP-sel, sE-sel and/or sL-sel) present within a subject (e.g. within the plasma) and/or to reduce the expression and/or activity of PSGL-1 (e.g., associated with endothelial dysfunction) can be identified and characterized by the compositions and methods of the present invention (e.g., for use a therapeutic for endothelial dysfunction). As used herein, the terms “levels of soluble selectin,” “levels of PSGL-1,” and “levels of a biomarker” refer to the amount of expression and/or activity of soluble selectin, PSGL-1 or other biomarker of the present invention. For example,

In one embodiment, the invention provides assays for screening candidate or test compounds that are substrates of a biomarker protein or polypeptide or a biologically active portion thereof. In another embodiment, the invention provides assays for screening candidate or test compounds that bind to or modulate the activity of a biomarker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (See, e.g., Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Libraries of compounds may be presented in solution (e.g., Houghten, Biotechniques 13:412-421 (1992)), or on beads (Lam, Nature 354:82-84 (1991)), chips (Fodor, Nature 364:555-556 (1993)), bacteria or spores (U.S. Pat. No. 5,223,409; herein incorporated by reference), plasmids (Cull et al., Proc. Nad. Acad. Sci. USA 89:18651869 (1992)) or on phage (Scott and Smith, Science 249:386-390 (1990); Devlin Science 249:404-406 (1990); Cwirla et al., Proc. Natl. Acad. Sci. 87:6378-6382 (1990); Felici, J. Mol. Biol. 222:301 (1991)).

In one embodiment, an assay is a cell-based assay in which a cell that expresses or is capable of generating a biomarker is contacted with a test compound, and the ability of the test compound to modulate biomarker presence, expression or activity is determined. Determining the ability of the test compound to modulate biomarker presence, expression or activity can be accomplished by monitoring, for example, changes in enzymatic activity or downstream products of expression (e.g., cellular integration and/or synaptic connectivity).

The ability of the test compound to modulate biomarker binding to a compound (e.g., a biomarker substrate or binding partner) can also be evaluated (e.g. the capacity of PSGL-1 binding to a substrate). This can be accomplished, for example, by coupling the compound (e.g., the substrate or binding partner) with a radioisotope or enzymatic label such that binding of the compound (e.g., the substrate) to a biomarker can be determined by detecting the labeled compound (e.g., substrate) in a complex.

Alternatively, the biomarker can be coupled with a radioisotope or enzymatic label to monitor the ability of a test compound to modulate biomarker binding to a biomarker substrate in a complex. For example, compounds (e.g., substrates) can be labeled with ¹²⁵I, ³⁵S ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a biomarker substrate) to interact with a biomarker with or without the labeling of any of the interactants can be evaluated. For example, a microphysiorneter can be used to detect the interaction of a compound with a biomarker without the labeling of either the compound or the biomarker (McConnell et al. Science 257:1906-1912 (1992)). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and a biomarker.

In yet another embodiment, a cell-free assay is provided in which a biomarker protein, or biologically active portion thereof, or nucleic acid is contacted with a test compound and the ability of the test compound to bind to the biomarker protein, or biologically active portion thereof, or nucleic acid is evaluated. Preferred biologically active portions of the biomarker proteins to be used in assays of the present invention include fragments that participate in interactions with substrates or other proteins (e.g., fragments with high surface probability scores).

Cell-free assays involve preparing a reaction mixture of the target gene protein and the test compound under conditions and for a time sufficient to allow the two components to interact and bind, thus forming a complex that can be removed and/or detected.

The interaction between two molecules (e.g., a biomarker protein and a test compound) can also be detected (e.g., using fluorescence energy transfer (FRET) (See, e.g., Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter).

In another embodiment, determining the ability of a biomarker to bind to a target molecule can be accomplished using real-time Biomolecular Interaction Analysis (BIA) (see, e.g., Sjolander and Urbaniczky, Anal. Chem. 63:2338-2345 (1991) and Szabo et al. Curr. Opin. Struct. Biol. 5:699-705 (1995)). “Surface plasmon resonance” or “BIA” detects biospecific interactions in real time, without labeling any of the interactants (e.g., BIACORE). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize biomarkers, an anti-biomarker antibody or its target molecule to facilitate separation of complexed from non-complexed forms of one or both of the molecules, as well as to accommodate automation of the assay. Binding of a test compound to a biomarker (e.g., protein or nucleic acid), or interaction of a biomarker with a target molecule in the presence and absence of a candidate compound, can be accomplished in any vessel suitable for containing the reactants. Examples of such vessels include microtiter plates, test tubes, and micro-centrifuge tubes.

For example, in one embodiment, a fusion protein can be provided which adds a domain that allows one or both of the molecules to be bound to a matrix. For example, glutathione-S-transferase-biomarker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or biomarker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of biomarkers binding or activity determined using standard techniques. Other techniques for immobilizing either biomarker molecule (e.g., nucleic acid or protein) or a target molecule on matrices include using conjugation of biotin and streptavidin. Biotinylated biomarker or target molecules can be prepared from biotin-NHS (N-hydroxy-succinimide) using techniques known in the art (e.g., biotinylation kit, Pierce Chemicals, Rockford, EL), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with biomarker or target molecules but which do not interfere with binding of the biomarker to its target molecule. Such antibodies can be derivatized to the wells of the plate, and unbound target or biomarkers trapped in the wells by antibody conjugation. Methods for detecting such complexes, in addition to those described above for the GST-immobilized complexes, include immunodetection of complexes using antibodies reactive with the biomarker or target molecule, as well as enzyme-linked assays which rely on detecting an enzymatic activity associated with the biomarker or target molecule.

Alternatively, cell free assays can be conducted in a liquid phase. In such an assay, the reaction products are separated from unreacted components, by any of a number of standard techniques, including, but not limited to: differential centrifugation (See, e.g., Rivas and Minton, Trends Biochem Sci 18:284-7 (1993)); chromatography (gel filtration chromatography, ion-exchange chromatography); electrophoresis (See, e.g., Ausubel et al., eds. Current Protocols in Molecular Biology 1999, J. Wiley: New York.); and immunoprecipitation (See, e.g., Ausubel et al., eds. Current Protocols in Molecular Biology 1999, J. Wiley: New York). Such resins and chromatographic techniques are known to one skilled in the art (See, e.g., Heegaard J. Mol. Recognit 11:141-8 (1998); Hageand Tweed J. Chromatogr. Biomed. Sci. Appl 699:499-525 (1997)). Further, fluorescence energy transfer may also be conveniently utilized, as described herein, to detect binding without further purification of the complex from solution.

The assay can include contacting the biomarker protein, or biologically active portion thereof, or nucleic acid with a known compound that binds the biomarker to form an assay mixture, contacting the assay mixture with a test compound, and determining the ability of the test compound to interact with a biomarker, wherein determining the ability of the test compound to interact with a biomarker includes determining the ability of the test compound to preferentially bind to biomarker protein, or biologically active portion thereof, or nucleic acid, or to modulate the activity of a target molecule, as compared to the known compound.

To the extent that biomarkers can, in vivo, interact with one or more cellular or extracellular macromolecules, such as proteins, inhibitors or inducers of such an interaction are useful. A homogeneous assay can be used to identify inhibitors.

For example, a preformed complex of the target gene product and the interactive cellular or extracellular binding partner product is prepared such that either the target gene products or their binding partners are labeled, but the signal generated by the label is quenched due to complex formation (See, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, biomarkers can be used as a “bait” in a two-hybrid assay or three-hybrid assay (See, e.g., U.S. Pat. No. 5,283,317; Zervos et al., Cell 72:223-232 (1993); Madura et al., J. Biol. Chem. 268.12046-12054 (1993); Bartel et al., Biotechniques 14:920-924 (1993); Iwabuchi et al., Oncogene 8:1693-1696 (1993); and Brent W0 94/10300; each of which is herein incorporated by reference), to identify proteins that bind to or interact with biomarkers (“biomarker-binding proteins” or “biomarker-bp”) and are involved in biomarker activity. Such biomarker-bps can be activators or inhibitors of signals by the biomarkers or targets as, for example, downstream elements of a biomarkers-mediated signaling pathway (e.g. synaptic activity (e.g. PKC)).

Modulators of biomarker expression can also be identified. For example, a cell or cell free mixture can be contacted with a candidate compound and the expression of biomarker nucleic acid (e.g., PSGL-1 DNA or mRNA) or protein evaluated relative to the level of expression of biomarker nucleic acid (e.g., DNA or mRNA) or protein in the absence of the candidate compound. When expression of biomarker nucleic acid (e.g., DNA or mRNA) or protein is greater in the presence of the candidate compound than in its absence, the candidate compound is identified as a stimulator of biomarker nucleic acid (e.g., DNA or mRNA) or protein expression. Alternatively, when expression of biomarker nucleic acid (e.g., DNA or mRNA) or protein is less (e.g., statistically significantly less) in the presence of the candidate compound than in its absence, the candidate compound is identified as an inhibitor of biomarker nucleic acid (e.g., DNA or mRNA) or protein expression. The level of biomarker nucleic acid (e.g., DNA or mRNA) or protein expression can be determined by methods described herein for detecting biomarker nucleic acid (e.g., DNA or mRNA) or protein.

A modulating agent can be identified using a cell-based or a cell free assay, and the ability of the agent to modulate the activity of a biomarker nucleic acid (e.g., DNA or mRNA) or protein can be confirmed in vivo, for example, in an animal such as an animal model for a disease (e.g., an animal model of vascular disease).

This invention further pertains to novel agents identified by the above-described screening assays. Accordingly, it is within the scope of this invention to further use an agent (e.g., test compound) identified as described herein (e.g., a biomarker modulating agent, an antisense biomarker nucleic acid molecule, a siRNA molecule, a biomarker specific antibody, or a biomarker-binding partner) in an appropriate animal model (such as those described herein) to determine the efficacy, toxicity, side effects, or mechanism of action, of treatment with such an agent. Furthermore, novel agents identified by the above-described screening assays can be, for example, used for treatments as described herein.

### IV Cell Therapies

In some embodiments, the present invention provides therapies for endothelial dysfunction (e.g., vascular disease (e.g., artherosclerosis). In some embodiments, therapies provide biomarkers and/or inhibitors of biomarkers (e.g., including but not limited to, PSGL-1 or inhibitors of PSGL-1 (e.g., PSGL-1 siRNA) for the treatment of endothelial cells (e.g., for decreasing inflammation, sclerosis or other events associated with endothelial dysfunction).

Therapeutics that Alter Biomarker Expression

In preferred embodiments, the present invention provides a method of inhibiting biomarker (e.g., PSGL-1, sP-sel, sE-sel and/or sL-sel) in a cell comprising altering biomarker (e.g., PSGL-1, sP-sel, sE-sel and/or sL-sel) expression and/or activity in the cell. In some embodiments, altering biomarker (e.g., PSGL-1, sP-sel, sE-sel and/or sL-sel) expression and/or activity comprises reducing biomarker (e.g., PSGL-1, sP-sel, sE-sel and/or sL-sel) expression and/or activity (e.g., thereby decreasing the serum level of sP-sel, sE-sel and/or sL-sel). In some embodiments, altering biomarker (e.g., PSGL-1) expression and/or activity comprises providing to the cell a composition comprising n biomarker (e.g., PSGL-1, sP-sel, sE-sel and/or sL-sel) inhibitor. The present invention is not limited by the type of biomarker inhibitor used to inhibit biomarker activity and/or expression for inhibiting biomarker in a cell. Indeed, any compound, pharmaceutical, small molecule or agent that can alter biomarker expression and/or activity is contemplated to be useful in the methods of the present invention. Examples of inhibitors of biomarker (e.g., PSGL-1, sP-sel, sE-sel and/or sL-sel) expression and/or activity that find use in treating (e.g., for delivering to and/or providing—e.g., expressing within) endothelial cells (e.g., associated with endothelial dysfunction (e.g., associated with vascular disease) include, but are not limited to, dominant-negative biomarker (e.g., PSGL-1, sP-sel, sE-sel and/or sL-sel) or derivative thereof, antisense nucleic acids (including, but not limited to, siRNAs, ribozymes and triple-helix-forming oligonucleotides), anti-biomarker antibodies (e.g., antibodies described herein, as well as intracellular single chain Fv antibodies.

In some embodiments, altering biomarker (e.g., PSGL-1, sP-sel, sE-sel and/or sL-sel) expression and/or activity comprises providing to a cell biomarker (e.g., PSGL-1, sP-sel, sE-sel and/or sL-sel) specific siRNAs. In some embodiments, the siRNAs reduce expression of biomarker. In some embodiments, altering biomarker (e.g., PSGL-1, sP-sel, sE-sel and/or sL-sel) expression and/or activity comprises providing to the cell an antibody specific for biomarker (e.g., PSGL-1, sP-sel, sE-sel and/or sL-sel). In some embodiments, the antibody reduces activity of biomarker (e.g., PSGL-1, sP-sel, sE-sel and/or sL-sel) in the cell. In some embodiments, altering biomarker (e.g., PSGL-1, sP-sel, sE-sel and/or sL-sel) expression and/or activity in the cell sensitizes the cell to therapeutic treatment. In some embodiments, sensitizing the cell to therapeutic treatment permits the cell to undergo treatment-induced cell death. In some embodiments, altering biomarker (e.g., PSGL-1, sP-sel, sE-sel and/or sL-sel) expression and/or activity inhibits symptoms of endothelial dysfunction (e.g., vascular disease).

In some embodiments, the present invention also provides a method of treating a subject with endothelial dysfunction comprising providing a composition comprising an inhibitor of biomarker (e.g., PSGL-1, sP-sel, sE-sel and/or sL-sel); and administering the composition to the subject under conditions such that symptoms associated with endothelial dysfunction are reduced.

In some embodiments, the present invention also provides a method of treating a subject with endothelial dysfunction (e.g., vascular disease) comprising providing a composition comprising an inhibitor of biomarker (e.g., PSGL-1, sP-sel, sE-sel and/or sL-sel); and administering the composition to the subject under conditions such that biomarker (e.g., PSGL-1, sP-sel, sE-sel and/or sL-sel) expression and/or activity is altered.

In some embodiments, the present invention provides methods and compositions suitable for therapy (e.g., drug, prodrug, pharmaceutical, or gene therapy) to alter biomarker (e.g., PSGL-1, sP-sel, sE-sel and/or sL-sel) gene expression, production, or function (e.g., to inhibit biomarker (e.g., PSGL-1, sP-sel, sE-sel and/or sL-sel) expression and/or activity).

In some embodiments, the present invention provides compositions comprising expression cassettes comprising a nucleic acid encoding an inhibitor of biomarker (e.g., PSGL-1, sP-sel, sE-sel and/or sL-sel) (e.g., siRNAs, peptides and the like), and vectors comprising such expression cassettes. The methods described below are generally applicable across many species. Any of the vectors and delivery methods disclosed herein can be used for modulation of biomarker (e.g., PSGL-1, sP-sel, sE-sel and/or sL-sel) activity (e.g., in a therapeutic setting). As disclosed herein, the therapeutic methods of the invention are optimally achieved by targeting the therapy to the affected cells. However, in another embodiment, a biomarker (e.g., PSGL-1, sP-sel, sE-sel and/or sL-sel) inhibitor can be targeted to cells, e.g., using vectors described herein in combination with well-known targeting techniques, for expression of biomarker (e.g., PSGL-1, sP-sel, sE-sel and/or sL-sel) modulators.

Furthermore, any of the therapies described herein can be tested and developed in animal models. Thus, the therapeutic aspects of the invention also provide assays for biomarker (e.g., PSGL-1) function.

In some embodiments, viral vectors are used to introduce biomarker inhibitors (e.g., siRNAs, proteins or fragments thereof, etc,) to cells. The present invention further provides a method for altering responsiveness of an endothelial dysfunctional cell to treatment comprising altering the levels of biomarker (e.g., PSGL-1) in the cell (e.g., through inhibiting biomarker (e.g., PSGL-1) expression using RNAi). The art knows well multiple methods of altering the level of expression of a gene or protein in a cell (e.g., ectopic or heterologous expression of a gene). The following are provided as exemplary methods of introducing biomarker (e.g., PSGL-1, sP-sel, sE-sel and/or sL-sel) inhibitors, and the invention is not limited to any particular method.

In some embodiments, the present invention provides a method of treating endothelial dysfunction comprising altering responsiveness of endothelial cells and/or platelets to treatment comprising making the endothelial cells and/or platelets either more or less responsive (e.g., sensitive) to the treatment. In some embodiments, making the endothelial cells and/or platelets more or less responsive (e.g., sensitive) to treatment comprises altering the level of biomarker (e.g., PSGL-1, sP-sel, sE-sel and/or sL-sel) expression and/or activity in the target cell. The present invention further provides a method of customizing endothelial cells and/or platelets for treatment by altering biomarker (e.g., PSGL-1, sP-sel, sE-sel and/or sL-sel) expression and/or activity in the cells. In some embodiments, altering the level of biomarker (e.g., PSGL-1, sP-sel, sE-sel and/or sL-sel) in the cell comprises introducing siRNA to the target cell (e.g., that inhibit biomarker (e.g., PSGL-1, sP-sel, sE-sel and/or sL-sel) expression)

While it is conceivable that a biomarker (e.g., PSGL-1, sP-sel, sE-sel and/or sL-sel) inhibitor (e.g., siRNA or peptide) may be delivered directly to a cell, a preferred embodiment involves providing a nucleic acid encoding a biomarker (e.g., PSGL-1, sP-sel, sE-sel and/or sL-sel) inhibitor to a cell. Following this provision, biomarker (e.g., PSGL-1, sP-sel, sE-sel and/or sL-sel) inhibitors are synthesized by the transcriptional and translational machinery of the cell. In some embodiments, additional components useful for transcription or translation may be provided by the expression construct comprising biomarker (e.g., PSGL-1, sP-sel, sE-sel and/or sL-sel) inhibitor sequence.

In some embodiments, the present invention provides methods for in vitro synthesis of biomarker inhibitors (e.g., siRNA, proteins or portions thereof) by in vitro transcription; such methods provide efficient and economical alternatives to chemical synthesis, and the biomarker inhibitors so synthesized can be used to transfect cells. In some embodiments, a siRNA construct (e.g., ds siRNA) can be designed to silence biomarker (e.g., PSGL-1), inserted into at least one expression cassette, and transfected into the cell in which the target gene (e.g., biomarker (e.g., PSGL-1)) is expressed. Furthermore, the present invention provides research applications wherein the effect of the lack of or disappearance of biomarker (e.g., PSGL-1) in the transfected cell is assessed; such results leading to elucidation of the function of the gene.

In some embodiments, research applications are in vivo in cells or tissues (e.g., as when cultured cells or tissues are transfected with either synthetic siRNA or siRNA expression constructs, as described above). In other embodiments, research applications are in vivo (e.g., as when organisms such as mammals are transfected with siRNA expression constructs, as described in further detail below).

In some embodiments, the nucleic acid encoding biomarker (e.g., PSGL-1) inhibitors (e.g., protein or siRNA) may be stably integrated into the genome of the cell. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on, among other things, the type of expression construct employed.

The ability of certain viruses to infect cells or enter cells via receptor-mediated endocytosis, and to integrate into host cell genome and express viral genes stably and efficiently have made them attractive candidates for the transfer of foreign genes into mammalian cells. In some embodiments, vectors of the present invention are viral vectors (e.g., phage or andenovirus vectors).

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein.

The expression vector may comprise a genetically engineered form of adenovirus. Knowledge of the genetic organization or adenovirus, a 36 kb, linear, double-stranded DNA virus, allows substitution of large pieces of adenoviral DNA with foreign sequences up to 7 kb (See Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 map units (m.u.)) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical adenovirus vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recombinant adenovirus and adeno-associated virus (see below) can both infect and transduce non-dividing human primary cells.

Adeno-associated virus (AAV) is an attractive vector system for use in the cell transduction of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994).

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the vector will be HSV. A factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and can be grown to high titers. Thus, delivery is less of a problem, both in terms of volumes needed to attain sufficient MOI and in a lessened need for repeat dosings.

In still further embodiments of the present invention, the nucleic acids to be delivered (e.g., nucleic acids encoding biomarker (e.g., PSGL-1) inhibitors) are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

In various embodiments of the invention, nucleic acid sequence encoding biomarker (e.g., PSGL-1) inhibitors is delivered to a cell as an expression construct. In order to effect expression of a gene construct, the expression construct must be delivered into a cell. As described herein, one mechanism for delivery is via viral infection, where the expression construct is encapsidated in an infectious viral particle. However, several non-viral methods for the transfer of expression constructs into cells also are contemplated by the present invention. In one embodiment of the present invention, the expression construct may consist only of naked recombinant DNA or plasmids (e.g., vectors comprising nucleic acid sequences of the present invention). Transfer of the construct may be performed by any of the methods mentioned which physically or chemically permeabilize the cell membrane. Some of these techniques may be successfully adapted for in vivo or ex vivo use, as discussed below.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

In certain embodiments of the present invention, the expression construct is introduced into the cell via electroporation. Electroporation involves the exposure of a suspension of cells (e.g., bacterial cells such as E. coli) and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

In other embodiments of the present invention, the expression construct is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells have been transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Further embodiments of the present invention include the introduction of the expression construct by direct microinjection or sonication loading. Direct microinjection has been used to introduce nucleic acid constructs into Xenopus oocytes (Harland and Weintraub, 1985), and LTK⁻ fibroblasts have been transfected with the thymidine kinase gene by sonication loading (Fechheimer et al., 1987).

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al., 1992; Curiel, 1994).

Still further expression constructs that may be employed to deliver nucleic acid construct to target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention.

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. In certain aspects of the present invention, the ligand will be chosen to correspond to a receptor specifically expressed on the EOE target cell population.

In other embodiments, the DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialganglioside, incorporated into liposomes and observed an increase in the uptake of the insulin gene by hepatocytes. It is contemplated that the tissue-specific transforming constructs of the present invention can be specifically delivered into the target cells in a similar manner.

In some embodiments, the present invention targets the expression of biomarker (e.g., PSGL-1). For example, in some embodiments, the present invention employs compositions comprising oligomeric antisense compounds, particularly oligonucleotides (e.g., those identified in the drug screening methods described above), for use in modulating the function of nucleic acid molecules encoding biomarker (e.g., PSGL-1), ultimately modulating the amount of biomarker (e.g., PSGL-1) expressed. This is accomplished by providing antisense compounds that specifically hybridize with one or more nucleic acids encoding biomarker (e.g., PSGL-1). The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of biomarker (e.g., PSGL-1). In the context of the present invention, “modulation” means either an increase (stimulation) or a decrease (inhibition) in the expression of a gene. For example, expression may be inhibited to potentially prevent tumor growth, angiogenesis and proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding biomarker (e.g., PSGL-1). The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂—(known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—(wherein the native phosphodiester backbone is represented as —O—P—O—CH₂—) of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃))₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 (1995)) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. degree ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-5-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

### Genetic Therapies

The present invention contemplates the use of any genetic manipulation for use in modulating the expression of biomarker (e.g., PSGL-1, sP-sel, sE-sel and/or sL-sel). Examples of genetic manipulation include, but are not limited to, gene knockout (e.g., removing the biomarker (e.g., PSGL-1) from the chromosome using, for example, recombination), expression of antisense constructs with or without inducible promoters, and the like. Delivery of nucleic acid construct to cells in vitro or in vivo may be conducted using any suitable method (e.g., using the methods described herein). A suitable method is one that introduces the nucleic acid construct into the cell such that the desired event occurs (e.g., expression of an antisense construct).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into endothelial cells or tissue and/or platelets using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

### Antibody Therapy

In some embodiments, the present invention provides antibodies (e.g., full length or portions thereof) that target biomarker (e.g., PSGL-1) expressing cells. In preferred embodiments, the antibodies used for therapy are humanized antibodies. In preferred embodiments, the antibody alters (e.g., inhibits) biomarker (e.g., PSGL-1) activity or function.

### Pharmaceutical Compositions

The present invention further provides pharmaceutical compositions (e.g., comprising biomarker (e.g., PSGL-1) inhibitors described herein). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (See U.S. Pat. No. 5,705,188, hereby incorporated by reference), cationic glycerol derivatives, and polycationic molecules, such as polylysine (See WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

In some embodiments, the invention provide pharmaceutical compositions containing (a) one or more biomarker (e.g., PSGL-1) inhibitors (e.g., antisense compounds) and (b) one or more other agents. Two or more combined agents (e.g., a statin and a biomarker (e.g., PSGL-1) inhibitor) may be used together or sequentially.

Dosing is dependent on severity and responsiveness of the disease state (e.g., vascular disease state) to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### Drug Screening Utilizing Transgenic Animals

The present invention provides methods and compositions for using transgenic animals (e.g., those described herein) as a target for screening drugs that can alter, for example, interaction between a biomarker (e.g., PSGL-1) and binding partners (e.g., p-Sel) or enhance or inhibit the activity of a biomarker (e.g., PSGL-1) or its signaling pathway. Drugs or other agents (e.g., test compounds (e.g., from a test compound library)) are exposed to the transgenic animal model and changes in phenotypes or biological markers are observed or identified. For example, in some embodiments, drug candidates are tested for the ability to alter sP-sel and/or sE-sel expression, presence and/or activity or function in PSGL-1 knockout or overexpressing animals. In some embodiments, test compounds are utilized to determine their ability to alter disease in a transgenic animal.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 (1994))); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (See, Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins (e.g. albumin), detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening.

### EXPERIMENTAL

The following examples are provided in order to demonstrate and further illustrate certain preferred embodiments and aspects of the present invention and are not to be construed as limiting the scope thereof.

### Example 1

### Materials and Methods

Mice. Psgl-1^{-/-} and Psel^{-/-} mice were purchased from Jackson Laboratory, Bar Harbor, Me. Breedings were performed between Psgl-1^{+/-} mice to produce Psgl-1^{-/-} and Psgl^{+/+} littermate controls for all comparisons. α(1,3)-fucosyltransferase-VII deficient mice (FucT-VII^{-/-}) have been described (See, e.g., Homeister et al., 2001. Immunity. 15:115-126) and compared to wild type mice from the same colony. All mice had previously been backcrossed several generations to the C57BL6/J background strain. In vivo sP-sel generation. Platelets isolated from wild-type mice were washed, activated with thrombin, and injected intravenously into mice as described (See, e.g., Berger et al., 1998. Blood 92:4446-4452). Retro-orbital blood samples were withdrawn under isoflurane anesthesia for the measurement of serum sP-sel immediately before injection and 60-minutes following injection.

Measurement of soluble selectins (P, E and L) and VCAM-1. Enzyme linked immunosorbent assays (R&D Systems, Inc., Minneapolis, Minn.) were used to determine the concentrations of soluble murine P-, E-, L-selectin, and VCAM-1 in mouse serum. Measurement of platelet P— selectin was performed as described (See, e.g., Kamath et al., 2002. Stroke 33:1237-1242) using Triton X100 on washed platelets followed by measurement of P-sel in the platelet supernatent.

Bone marrow transplantation. Bone marrow transplantation experiments from Psgl-1^{-/-} or Psgl-1^{+/+} donors to irradiated Psgl-1^{+/+} recipients were performed as described (See, e.g., Eitzman et al., 2003. J. Am. Soc. Nephrol. 14:298-302).

### Example 2

### PSGL-1 Regulates the Generation of sP-sel

During development of the present invention, it was determined whether a cellular blood component may play a role in the generation of soluble selectins (e.g., soluble P selectin (sP-sel)). A 100-kD sP-sel fragment is generated following injection of activated, but not resting, wild-type platelets into P-sel deficient mice (See, e.g., Berger et al., 1998. Blood 92:4446-4452). In vitro, a loss of labeled surface platelet P-sel was generated following incubation of activated platelets with whole blood but not plasma (See, e.g., Berger et al., 1998. Blood 92:4446-4452). The primary ligand for P-sel, PSGL-1, is expressed on leukocytes. Thus, experiments were conducted to determine whether PSGL-1 played a role in regulating sP-sel concentrations. The in vivo levels of sP-sel were determined in mice deficient in PSGL-1.

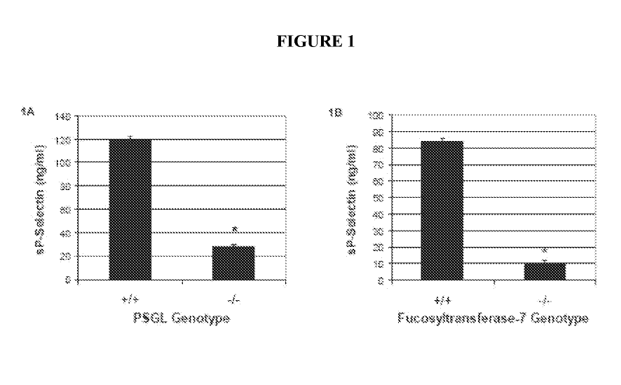

Serum samples from Psgl-1^{+/+} mice displayed 4.6-fold greater sP-sel than serum from Psgl-1^{-/-} mice (See FIG. 1a). Surprisingly, total platelet P-sel levels were increased in Psgl-1^{-/-} mice compared to wild-type mice (1.97±0.06 vs 1.56±0.09 ng/ml, p<0.05). Thus, the present invention provides that deficiency of PSGL-1 is not associated with total P-sel deficiency, but rather PSGL-1 is specifically involved in regulating the generation of sP-sel. Also surprising was the fact that sP-sel concentrations were not different between Psgl-1^{+/-} and Psgl-1^{+/+} mice (123±3.3 vs 116±2.3 ng/mL), providing that in the unchallenged state, a 50% reduction in PSGL-1 alleles does not affect sP-sel generation.

### Example 3

### Fucosylation of PSGL-1 is Important for PSGL-1's Ability to Regulate sP-sel Levels

The capacity for physiologic ligand binding to selectins is regulated by α(1,3)-fucosylation (See, e.g., Homeister et al., 2001. Immunity. 15:115-126; Maly et al., 1996. Cell 86:643-653). For example, fucosylation of PSGL-1 by the myeloid α(1,3)-fucosytransferase-TVII is required for selectin binding (See, e.g., Huang et al., 2000. J. Biol. Chem. 275:31353-31360). It was determined whether fucosylation of PSGL-1 impacted serum levels of soluble selectins. As shown in FIG. 1b, serum from FucT-VII^{+/+} mice contained 7.5-fold more sP-sel than serum from FucT-VII^{-/-} mice. Although an understanding of the mechanism is not necessary to practice the present invention and the present invention is not limited to any particular mechanism of action, in some embodiments, ligand binding to P-sel (e.g., dependent upon the fucosylation of the ligand (e.g., PSGL-1)) is involved in the generation of sP-sel.

### Example 4

### PSGL-1 Status Following Platelet Challenge

Since P-sel has been shown to mediate the formation of platelet-leukocyte aggregates following injection of activated platelets (See, e.g., Huo et al., 2003. Nat. Med. 9:61-67), the effect of PSGL-1 status on the generation of sP-sel was determined following a platelet challenge. After injection of thrombin-activated wild-type platelets, sP-sel increased by 16.6±7.6 ng/ml in Psgl-1^{+/+} mice, whereas sP-sel decreased by 7.5±2.6 ng/ml in Psgl-1^{-/-} mice (p=0.039). Although an understanding of the mechanism is not necessary to practice the present invention and the present invention is not limited to any particular mechanism of action, in some embodiments, platelets are a source of soluble selectins (e.g., sP-sel).

### Example 5

### Characterization of Tissue Sources of Soluble Selectins Regulated by PSGL-1

In addition to platelets being a source of sP-sel levels (See Example 4), the endothelium may also be a primary contributor to sP-sel levels in Psgl-1^{+/+} mice. For example, it has been demonstrated, using bone marrow transplantation techniques, that the predominant source of sP-sel in atherosclerotic-prone mice was the endothelium with only a minor contribution from platelets (See, e.g., Burger and Wagner. 2003. Blood 101:2661-2666).

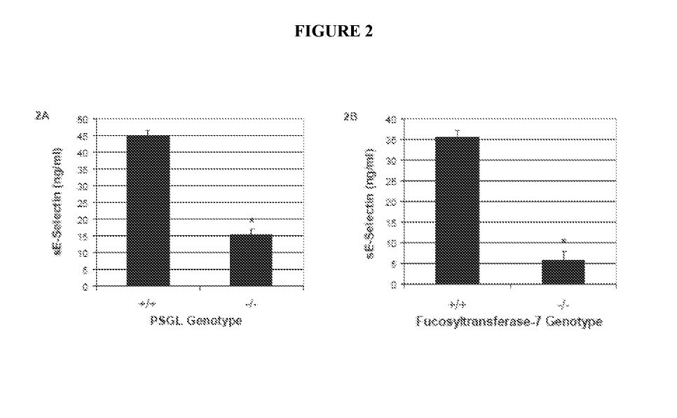

Thus, in order to characterize potentially relevant tissue sources of soluble selectins regulated by PSGL-1, concentrations of the soluble endothelial-specific selectin, sE-sel, were measured in Psgl-1^{+/+} and Psgl-1^{-/-} mice. Serum from Psgl-1^{+/+} mice contained 3.2-fold greater sE-sel than serum from Psgl-1^{-/-} mice (See FIG. 2a). Similarly, serum from FucT-VII^{+/+} mice contained 6-fold greater sE-sel than serum from FucT-VII^{-/-} mice (FIG. 2b). Thus, sE-sel levels are dependent on PSGL-1 expression and α(1,3)-fucoslyation. Circulating levels of vascular cell adhesion molecule-1, an endothelial adhesion molecule with expression that is under regulatory influences similar to those of E-sel (See, e.g., Cernuda-Morollon and Ridley. 2006. Circ. Res. 98:757-767), were not different between Psgl-1^{-/-} and Psgl-1^{+/+} mice (947±49 vs. 913±49 ng/mL). Although an understanding of the mechanism is not necessary to practice the present invention and the present invention is not limited to any particular mechanism of action, in some embodiments, expression of PSGL-1 and PSGL-1 binding capacity (e.g., enabled by fucosylation) are involved in the generation of soluble selectins (e.g., sE-sel).

The endothelial-specific expression of E-sel indicates that the endothelial cell is the source of PSGL-1-dependent sE-sel generation. However, while PSGL-1 is a major ligand for P-sel, it may be a relatively minor E-sel ligand (See, e.g., Zanardo et al., 2004. Blood 104:3766-3773). Therefore, to examine whether generation of sE-sel is due to a direct interaction of PSGL-1 with E-sel or secondary to a facilitory role of PSGL-1 with P-sel, sE-sel levels were measured in P-sel^{-/-} mice. sE-sel levels in P-sel^{-/-} mice were reduced to similar levels as those observed in Psgl-1^{-/-} mice (14.6±3.7 vs. 15.3±1.9 ng/ml, respectively). Although an understanding of the mechanism is not necessary to practice the present invention and the present invention is not limited to any particular mechanism of action, in some embodiments, generation of sE-sel is due to ligands other than PSGL-1, and interaction of these other ligands with E-sel may require initial interactions between PSGL-1 and P-sel. For example, it has previously been shown that leukocyte rolling on E-sel is reduced following treatment with a mAb to block P-sel function (See, e.g., Xia et al., 2002. J. Clin. Invest 109:939-950).

In some embodiments, sP-sel and sE-sel may be generated during leukocyte interactions with the endothelium, such as rolling, and selectin shedding may actually be required for efficient rolling. L-selectin shedding has been previously shown to occur rapidly during the process of leukocyte rolling in an in vitro hydrodynamic flow model, and inhibition of the shedding process with a metalloprotease inhibitor reduced neutrophil rolling leading to increased neutrophil accumulation (See, e.g., Walcheck et al., 1996. Nature 380:720-723).

To determine if PSGL-1 affected generation of sL-sel, sL-sel was measured in Psgl-1^{+/+} and Psgl-1^{-/-} mice. sL-sel was increased in Psgl-1^{-/-} mice compared to Psgl^{+/+} mice (1.85±0.068 vs 1.63±0.030 ug/ml, p=0.01). Although an understanding of the mechanism is not necessary to practice the present invention and the present invention is not limited to any particular mechanism of action, in some embodiments, in the absence of PSGL-1-dependent endothelial selectin binding, there are increased interactions of leukocyte L-sel with other endothelial ligands, resulting in increased generation of sL-sel.

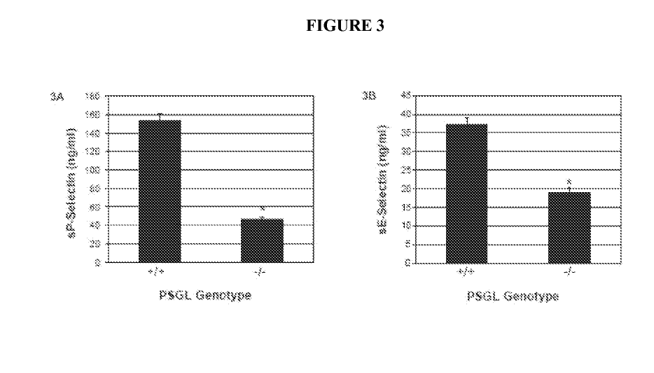

In addition to leukocytes, PSGL-1 has also been shown to be present on endothelial cells and to bind P-sel (See, e.g., Rivera-Nieves et al., 2006. J. Exp. Med. 203:907-917). In order to determine the relevant PSGL-1 tissue compartment for generation of sP-sel, bone marrow transplantation from Psgl-1^{-/-} into Psgl-1^{+/+} mice was performed. sP-sel levels were 3.3-fold lower and sE-sel were 2.0-fold lower compared to transplanted wild-type controls, demonstrating that the relevant PSGL-1 pool for generating soluble selectins is bone marrow-derived (See FIGS. 3a and b).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A method for detecting endothelial dysfunction in a subject, comprising:
a) providing a subject; and
b) detecting expression and/or activity of PSGL-1 in said subject.

2. The method of claim 1, wherein detecting PSGL-1 comprises detecting PSGL-1 nucleic acid.

3. The method of claim 1, wherein detecting PSGL-1 comprises detecting PSGL-1 protein.

4. The method of claim 3, comprising detecting fucosylation of PSGL-1.

5. The method of claim 1, further comprising detecting soluble P-selectin.

6. The method of claim 1, further comprising detecting soluble E-selectin.

7. The method of claim 1, further comprising detecting expression of a protein selected from the group consisting of soluble ICAM-1, soluble VCAM-1, soluble thrombomodulin, and von Willebrand factor.

8. The method of claim 1, wherein elevated expression and/or activity of PSGL-1 in said subject compared to a healthy subject is correlated with the presence of endothelial dysfunction, and wherein said endothelial dysfunction is associated with a disease selected from the group consisting of hypertension, hypercholesterolaemia, vascular disease and diabetes.

9. The method of claim 8, wherein said vascular disease is selected from the group consisting of atherosclerosis, artery disease, vascular disease, cardiovascular disease, restinosis, stenosis, occlusion, hemostatic disorder, coronary artery disease, stroke, heart attack, and diabetes mellitus.

10-11. (canceled)

12. A method for characterizing the efficacy of therapeutic drug treatment comprising:
a) providing a subject;
b) determining the expression level of PSGL-1 in said subject prior to said treatment;
c) administering said treatment to said subject; and
d) determining the expression level of PSGL-1 in said subject subsequent to said treatment.

13. The method of claim 12, wherein said subject is selected from the group consisting of a subject at risk for endothelial dysfunction and a subject suffering from endothelial dysfunction.

14. The method of claim 12, further comprising detecting the level of soluble P selectin in said subject prior to said treatment.

15. The method of claim 12, further comprising detecting the level of soluble P selectin in said subject subsequent to said treatment.

16. The method of claim 12, further comprising detecting the level of soluble E selectin in said subject prior to said treatment.

17. The method of claim 12, further comprising detecting the level of soluble E selectin in said subject subsequent to said treatment.

18. The method of claim 12, wherein detection of a decrease in levels of soluble P selectin or soluble E selectin in said subject subsequent to said treatment is indicative of a favorable response to said treatment.

19-25. (canceled)

26. A method for determining a course of treatment in a subject comprising:
a) providing a subject;
b) determining the expression level of PSGL-1 in said subject; and
c) identifying a course of treatment for said subject based upon said expression level of PSGL-1 in said subject.

27. The method of claim 26, further comprising:
d) determining the expression level of PSGL-1 in said subject subsequent to administering said treatment to said subject.

28. The method of claim 26, wherein said subject is selected from the group consisting of a subject at risk for endothelial dysfunction and a subject suffering from endothelial dysfunction.

29. The method of claim 26, further comprising detecting the level of soluble P selectin in said subject.

30. The method of claim 26, further comprising detecting the level of soluble E selectin in said subject.

31. The method of claim 27, further comprising:
e) determining the expression level of soluble P selectin and/or soluble E selectin in said subject subsequent to administering said treatment to said subject.

32. The method of claim 31, wherein detection of a decrease in levels of soluble P selectin and/or soluble E selectin in said subject subsequent to said treatment is indicative of a favorable response to said treatment.

33. The method of claim 26, wherein said course of treatment is a treatment for a disease selected from the group consisting of hypertension, hypercholesterolaemia, vascular disease and diabetes.

34. (canceled)
